(19) United States
(12) Patent Application Publication
(10) Pub. No.: US 2005/0065207 A1
(43) Pub. Date: Mar. 24, 2005

(54) Method for the preparation of escitalopram

(76) Inventors: Michael B Sommer, Bagsvaerd (DE); Ole Nielsen, Valby (DE); Hans Petersen, Vanlose (DE); Haleh Ahmadian, Solrod Strand (DE); Henrik Pedersen, Bronshoj (DE); Peter Brosen, Herlev (DE); Fiona Geiser, Glen Mills, PA (US); James Lee, Philadelphia, PA (US); Geoffrey Cox, West Chester, PA (US); Olivier Dapremont, Folsom, CA (US); Christina Suteu, Illkirch (FR); Sebastian P Assenza, Fort Salonga, NY (US); Shankar Hariharan, Smithtown, NY (US); Usha Nair, Nashville, TN (US)

Correspondence Address: DARBY & DARBY P.C., P. O. BOX 5257, NEW YORK, NY, 10150-5257, US

(21) Appl. No.: 10/483,824

(22) PCT Filed: Jul. 12, 2002
(86) PCT No.: PCT/DK02/00491

(30) Foreign Application Priority Data
Jul. 13, 2001 (DK) PA 2001 01101
Dec. 11, 2001 (DK) PA 2001 01851
Dec. 11, 2001 (DK) PA 2001 01852

(51) Int. Cl. C07D 307/87; A61K 31/343

(52) U.S. Cl. 514/469; 549/467

(57) ABSTRACT

A novel method is provided for the manufacture of escitalopram. The method comprises chromatographic separation of the enantiomers of citalopram or an intermediate in the production of citalopram using a chiral stationary phase such as Chiralpak™ or Chiralcel™ OD. Novel chiral intermediates for the synthesis of Escitalopram made by said method are also provided.

### FIELD OF INVENTION

The present invention relates to the preparation of the compound escitalopram, which is the S-enantiomer of the well-known antidepressant drug citalopram, i.e. (S)-1-[3-(dimethylamino)propyl]-1-(4-fluorophenyl)-1,3-dihydro-5-isobenzofuran-carbonitrile, or a pharmaceutically acceptable salt thereof for the preparation of pharmaceutical preparations.

### BACKGROUND OF THE INVENTION

Citalopram is a well-known antidepressant drug that has now been on the market for some years and has the following structure: 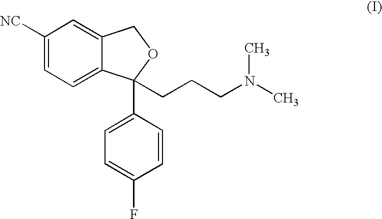
It is a selective, centrally acting serotonin (5-hydroxytryptamine; 5-HT) reuptake inhibitor, accordingly having antidepressant activities.

Citalopram was first disclosed in DE 2,657,013, corresponding to U.S. Pat. No. 4,136,193. This patent publication i.a. outlines a process for the preparation of citalopram from the corresponding 5-bromo-derivative by reaction with cuprous cyanide in a suitable solvent. Further processes for the preparation of citalopram by exchange of 5-halogen or CF₃—(CF₂)ₙ—SO₂—O—, n being 0-8, with cyano are disclosed in WO 0011926 and WO 0013648.

The diol of formula II, 4-[4-(dimethylamino)-1-(4′-fluorophenyl)-1-hydroxy-1-butyl]-3-(hydroxymethyl)-benzonitrile, and its use as an intermediate in the preparation of citalopram has been disclosed in e.g. U.S. Pat. No. 4,650,884. 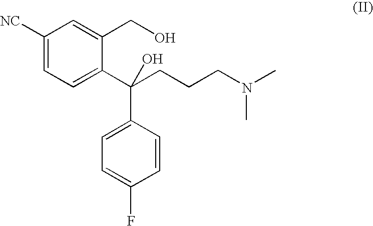
Escitalopram, the enantiomers of the diol II and methods for their preparation are disclosed in U.S. Pat. No. 4,943,590. Two routes to escitalopram are disclosed, both of them are starting with the racemic diol II. In the first route, the diol II is reacted with an enantiomerically pure acid derivative, such as (+) or (−)-α-methoxy-α-trifluoromethyl-phenylacetyl chloride to form a mixture of diastereomeric esters, which are separated by HPLC or fractional crystallization, whereupon the ester with the right stereochemistry is enantioselectively converted into escitalopram. In the second route, the diol II is separated into the enantiomers by stereoselective crystallization with an enantiomerically pure acid such as (+)-di-p-toluoyltartaric acid, whereupon the S-enantiomer of the diol II is enantioselectively converted to escitalopram. Both of these routes involve consumption of expensive, enantiomerically pure reagents and give relatively low yields resulting in that they are economically and environmentally infeasible for industrial production. The stereoselectivity of the pharmacological action of citalopram, i.e. the 5-HT-reuptake inhibition residing in the S-enantiomer, and accordingly, the antidepressant effect of said enantiomer is also disclosed in U.S. Pat. No. 4,943,590. Escitalopram has now been developed as an antidepressant. Hence, there is a desire for an improved method for preparation of escitalopram.

It is known to those skilled in the art that two enantiomers in certain situations may be separated by liquid chromatography using a chiral stationary phase. The chiral stationary phase has to be found by screening of the available chiral stationary phases for one, which is effective in separating the pair of enantiomers in question, and there may not always be an available chiral stationary phase suitable for the purpose.

Conventional liquid chromatography is a batch process consuming large amounts of solvents and, hence, is generally not economically feasible for industrial production. Chromatographic processes, which are advantageous by being continuous and generally consuming reduced amounts of solvents, are known to those skilled in the art. Simulated moving bed (SMB) chromatography is one such continuous chromatographic process.

EP 563,388 discloses a simulated moving bed (SMB) chromatographic process wherein enantiomers of an optically active compound are separated and the stationary phase comprises silica gel coated with a chiral material such as a cellulose ester.

Hence, there is a desire for a chiral stationary phase which is effective in separating the enantiomers of citalopram, or a compound which is an intermediate in the manufacture of citalopram.

There is no method which enables one, a priori, to forecast which chiral stationary phase will be effective in separating a given pair of enantiomers. The chiral stationary phase for separation of a pair of enantiomers has to be found by laborious testing of chiral stationary phases selected from the vast amount of available chiral stationary phases.

### OBJECTS OF THE INVENTION

One object of the invention is to provide a novel and economically feasible chromatographic method for separating the enantiomers of citalopram, or a compound which is an intermediate in the manufacture of citalopram.

Another object of the invention is to provide novel optically resolved intermediates for the manufacture of escitalopram.

### SUMMARY OF THE INVENTION

As used herein, the terms ‘separation of enantiomers’ and ‘separation into enantiomers’ refer to any process resulting in two or more fractions wherein the ratio between the two enantiomers deviates from 1:1. The term ‘optically resolved’ refers to the product of any such process.

As used herein, the term ‘purity’ means the purity of the enantiomer measured as percent enantiomeric excess (ee).

As used herein, the term ‘carbohydrate derivative’ means any compound which principally can be derived from a carbohydrate by substitution of one or more hydroxyl groups with another substituent leaving the stereochemical structure intact.

As used herein, the terms ‘intermediate for the manufacture of escitalopram’ and ‘intermediate compounds in the preparation of citalopram’ means any intermediate in any known process for the manufacture of escitalopram.

Throughout the application, structural formula of chiral compounds refer to the racemates if the stereochemistry is not indicated.

Laborious experimentation has now resulted in a new and inventive process for the manufacture of escitalopram comprising separation of the enantiomers of citalopram or an intermediate in the manufacture of citalopram by chromatography using a chiral stationary phase.

Accordingly, the present invention relates to a novel process for the preparation of escitalopram having the formula 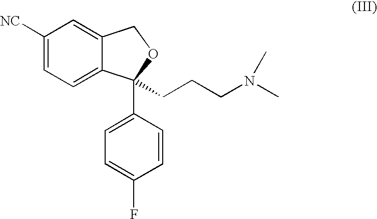
comprising preparation of a compound of formula 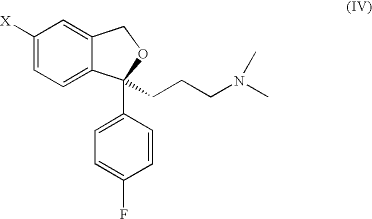
wherein X is a cyano group, halogen or any other group which may be converted to a cyano group by optical resolution by chromatography of the racemic compound of formula 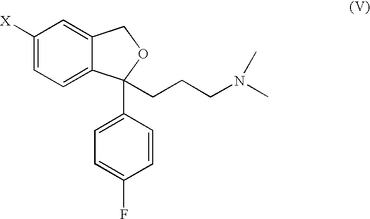
wherein X is as defined above; and if X is not a cyano group, then followed by conversion of X to a cyano group and thereafter isolation of escitalopram or a pharmaceutically acceptable salt thereof.

In one preferred embodiment of the invention, citalopram is separated into its enantiomers by chromatography using a chiral stationary phase.

Accordingly the present invention relates to a novel process for the preparation of escitalopram having the formula 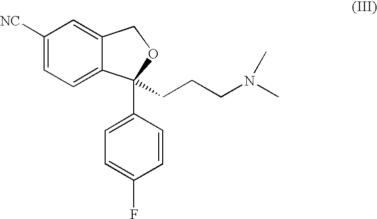
comprising optical resolution by chromatography of a compound of formula 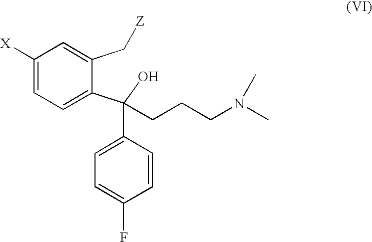
wherein X is a cyano group, halogen or any other group that may be converted to a cyano group and Z is hydroxy or a leaving group, to form the compound of formula 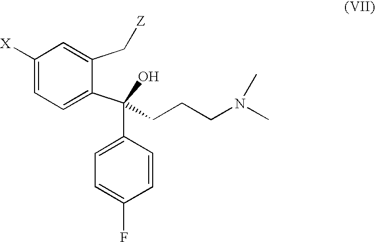
and if Z is OH conversion of the group Z to a leaving group and then ring closure of the resulting compound of formula (VII) wherein Z is a leaving group to form a compound of formula 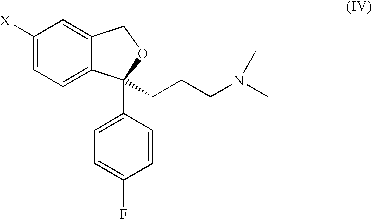
wherein X is as defined above, and if X is not a cyano group, then followed by conversion of the group X in the compound of formula (III) to a cyano group, followed by isolation of escitalopram or a pharmaceutically acceptable salt thereof.

In another preferred embodiment of the invention, the intermediate diol II 4-[4-(dimethylamino)-1-(4′-fluorophenyl)-1-hydroxy-1-butyl]-3-(hydroxymethyl)-benzo-nitrile is separated into its enantiomers by chromatography using a chiral stationary phase. The obtained (S)-4-[4-(dimethylamino)-1-(4′-fluorophenyl)-1-hydroxy-1-butyl]-3-(hydroxymethyl)-benzonitrile may be transformed into escitalopram by methods known to those skilled in the art, such as treatment with para-toluensulfonylchloride and a base, e.g. triethylamine, as disclosed in U.S. Pat. No. 4,943,590.

The invention also relates to the intermediate having the formula 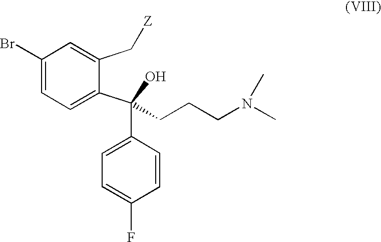
wherein Z is as defined above.

In a further embodiment, the present invention relates to the S-enantiomer of 5-Br-citalopram having the formula 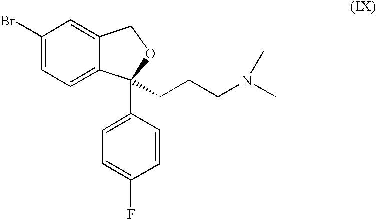
or salts thereof.

The racemic compounds of formula (V) and (VI) may be resolved by liquid chromatography or super or sub critical chromatography using a chiral stationary phase.

The chiral stationary phase may comprise an optically active high molecular compound, e.g. a polysaccharide derivative, such as esters or carbamates of cellulose or amylose, a polyacrylate derivative (e.g. a methacrylate derivative, such as poly(triphenylmethylmethacrylate)) or a polyamide derivative, a protein with an asymmetric or disymmetric chain (bovine serum albumin bonded to silica, cellulase covalently bonded to aldehyde silica), polymers with an asymmetric centre in its side chains etc. . . .

Another possibility is a chiral stationary phase comprising a low molecular compound having optical resolution capability, e.g. crown ethers ((S) or (R)-18-crown-6-ether on silica) and cyclodextrin derivatives (alpha cyclodextrin bonded to silica).

Other important chiral separation factors which may be comprised by the chiral stationary phase are amino acids and derivatives thereof, esters or amids of amino acids, acetylated amino acids and oligopeptides.

Still another possibility is a particulate polysaccharide material, e.g microcrystalline cellulose triacetate.

Chiral stationary phases including polysaccharide derivatives and polyamides useful for separation of enantiomers are described in EP 0 147 804, EP 0 155 637, EP 0 157 365, EP 0 238 044, WO 95/18833, WO 97/04011, EP 0656 333 and EP 718 625.

Particles of polysaccharides useful for the separation of optical enantiomers are described in EP 0706 982.

Preferably, the chiral stationary phase comprises a carbohydrate derivative, more preferred a polysaccharide derivative and most preferred an amylose or cellulose derivative.

Suitably, the polysaccharide adsorbed on the silica gel carry groups such as phenylcarbamoyl, 3,5-dimethyl-phenylcarbamoyl, 4-chlorophenylcarbamoyl, 3,5-dichloro-phenylcarbamoyl, acetyl, benzoyl, cinnamoyl, 4-methyl-benzoyl or S-alpha-phenylethyl carbamoyl.

Preferably, the carbohydrate derivative comprises phenyl carbamate substituents, which optionally may be substituted with one or more C₁₋₄-alkyl groups, preferably methyl groups.

The chiral compound, which is the chiral separating factor of the stationary phase, may suitably be adsorbed on a carrier, such as silica gel.

Suitably, the chiral stationary phase is Chiralpak™ AD, a silica gel supported amylose derivative wherein the majority of the hydroxyl groups are substituted with 3,5-dimethylphenyl carbamate groups, or Chiralcel™ OD, a silica gel supported cellulose derivative wherein the majority of the hydroxyl groups are substituted with 3,5-dimethylphenyl carbamate groups. Chiralpak™ AD and Chiralcel™ OD are both obtainable from Daicel Chemical Industries Ltd.

Chiral stationary phases comprising amylose phenyl carbamate derivatives are especially suitable for resolvation of compounds of formula (VI). Exemplary of such chiral stationary phases is Chiralpak™ AD.

Chiral stationary phases comprising cellulose phenyl carbamate derivatives are especially suitable for resolvation of compounds of formula (V). Exemplary of such chiral stationary phases is Chiralcel™ OD.

The nature of the substituent X has little influence on the resolvation of the compounds as it is distant from the chiral center.

Any liquid chromatographic separation method may be used for the separation of the enantiomers. Preferably, the chromatographic separation method comprises a continuous chromatographic technology, suitably simulated moving bed technology.

The eluent is typically selected from the group comprising acetonitrile, alcohols, such as methanol, ethanol or isopropanol, and alkanes, such as cyclohexane, hexane or heptane, and mixtures thereof. An acid such as formic acid, acetic acid and trifluoroacetic acid and/or a base such as diethylamine, triethylamine, propylamine, isopropylamine and dimethyl-isopropyl-amine may be added to the eluent.

Alternatively, super or sub critical carbon dioxide containing a modifier may be used as eluent. The modifier is selected from lower alcohols such as methanol, ethanol, propanol and isopropanol. An amine, such as diethylamine, triethylamine, propylamine, isopropylamine and dimethyl-isopropyl-amine and optionally an acid, such as formic acid, acetic acid and trifluoroacetic acid may be added.

Suitably, the chromatographic method used is a liquid chromatographic method.

A suitable eluent according to this embodiment of the invention is acetonitrile.

Another suitable eluent according to this embodiment of the invention is a mixture of iso-hexane and isopropanol. A suitable mixture contains iso-hexane 98% vol and isopropanol 2% vol.

Another suitable eluent according to the invention is super or sub critical carbon dioxide containing 10% vol methanol with 0.5% vol diethylamine and 0.5% vol trifluoroacetic acid.

One embodiment of the invention comprises novel optically resolved intermediates for the manufacture of escitalopram.

When Z is OH in the compound of formula (VII), the alcohol group, Z, may be converted to a suitable leaving group such as a sulfonate ester or a halide. The former is carried out by reaction with sulfonyl halides, such as methanesulfonyl chloride and p-toluensulfonyl chloride. The latter is achieved by reaction with halogenating agents such as thionyl chloride or phosphorus tribromide.

Ring closure of the compounds of formula (VII), wherein Z is a leaving group, such as a sulfonate ester or halogen may thereafter be carried out by treatment with a base such as KOC(CH₃)₃ or other alkoxides, NaH or other hydrides, triethylamine, ethyldiisopropylamine or pyridine in an inert organic solvent, such as tetrahydrofuran, toluene, DMSO, DMF, t-butyl methyl ether, dimethoxyethane, dimethoxymethane, dioxane, acetonitrile or dichloromethane.

The ring closure is analogous to the process described in U.S. Pat. No. 4,943,590.

The compound of formula (IV) may be converted to escitalopram having the formula 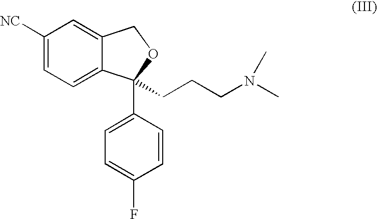
by a number of methods as described below.

As mentioned above, X in the compound of formula (IV) may be a cyano group, halogen, preferably chloro or bromo, or any other compound which may be converted to a cyano group.

Such other groups, X, which may be converted to a cyano group may be selected from the groups of formula CF₃—(CF₂)ₙ—SO₂—O—, wherein n is 0-8, —OH, —CHO, —CH₂OH, —CH₂NH₂, —CH₂NO₂, —CH₂Cl, —CH₂Br, —CH₃, —NHR¹, —COOR², —CONR²R³, wherein R¹ is hydrogen or alkylcarbonyl, and R² and R³ are selected from hydrogen optionally substituted alkyl, aralkyl or aryl,
and a group of formula 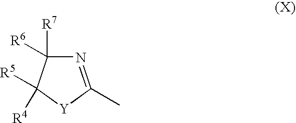
wherein Y is O or S;

R⁴-R⁵ are each independently selected from hydrogen and C₁₋₆ alkyl or R⁴ and R⁵ together form a C₂₋₅ alkylene chain thereby forming a spiro ring; R⁶ is selected from hydrogen and C₁₋₆ alkyl, R⁷ is selected from hydrogen, C₁₋₆ alkyl, a carboxy group or a precursor group for a carboxy group, or R⁶ and R⁷ together form a C₂₋₅ alkylene chain thereby forming a spiro ring.

When X is halogen, in particular bromo or chloro, conversion of the compound of formula (IV) to form escitalopram may be carried out according to the procedures described in U.S. Pat. No. 4,136,193, WO 00/13648, WO 00/11926 and WO 01/02383 or other procedures suitable for such conversions.

According to U.S. Pat. No. 4,136,193, conversion of the 5-bromo group may be carried out by reaction of a compound of formula (IV) wherein X is bromo, with CuCN.

WO 00/13648 and WO 00/11926 describes the conversion of a 5-halogen or a triflate group to a cyano group by cyanation with a cyanide source in presence of a Pd or Ni catalyst.

The cyanide source used according to the catalysed cyanide exchange reaction may be any useful source. Preferred sources are KCN, NaCN or (R′)₄NCN, where (R′)₄ indicates four groups which may be the same of different and are selected from hydrogen and straight chain or branched C₁₋₆ alkyl.

The cyanide source is used in stoichiometric amount or in excess, preferably 1-2 equivalents are used pr. equivalent starting material. (R′)₄N³⁰ may conveniently be (Bu)₄N³⁰ . The cyanide source is preferably NaCN or KCN or Zn(CN)₂.

The palladium catalyst may be any suitable Pd(0) or Pd(II) containing catalyst, such as Pd(PPh₃)₄, Pd₂(dba)₃, Pd(PPh)₂Cl₂, etc. The Pd catalyst is conveniently used in an amount of 1-10, preferably 2-6, most preferably about 4-5 mol %.

In one embodiment, the reaction is carried out in the presence of a catalytic amount of Cu⁺ or Zn²⁺. Catalytic amounts of Cu⁺ and Zn²⁺, respectively, means substoichiometric amounts such as 0.1-5, preferably 1-3 mol. Conveniently, about ½ eq. is used per eq. Pd. Any convenient source of Cu⁺ and Zn⁺⁺ may be used. Cu⁺ is preferably used in the form of CuI, and Zn²⁺ is conveniently used as the Zn(CN)₂ salt.

In a preferred embodiment, cyanation is carried out by reaction with ZnCN₂ in the presence of a Palladium catalyst, preferably Pd(PPh₃)₄ (tetrakis(triphenylphos-phine)palladium).

The nickel catalyst may be any suitable Ni(0) or Ni(II) containing complex which acts as a catalyst, such as Ni(PPh₃)₃, (σ-aryl)-Ni(PPh₃)₂Cl, etc. The nickel catalysts and their preparation are described in WO 96/11906, EP-A-613720 and EP-A-384392.

In a particularly preferred embodiment, the nickel(0) complex is prepared in situ before the cyanation reaction by reduction of a nickel(II) precursor such as NiCl₂ or NiBr₂ by a metal, such as zinc, magnesium or manganese in the presence of excess of complex ligands, preferably triphenylphosphin.

The Ni-catalyst is conveniently used in an amount of 0.5-10, preferably 2-6, most preferably about 4-5 mol %.

In one embodiment, the reaction is carried out in the presence of a catalytic amount of Cu⁺ or Zn²⁺.

Catalytic amounts of Cu⁺ and Zn²⁺, respectively, means substoichiometric amounts such as 0.1-5, preferably 1-3%. Any convenient source of Cu⁺ and Zn²⁺ may be used. Cu⁺ is preferably used in the form of CuI and Zn²⁺ is conveniently used as the Zn(CN)₂ salt or formed in situ by reduction of a nickel (II) compounds using zinc.

The cyanation reaction may be performed neat or in any convenient solvent, such solvent includes DMF, NMP, acetonitril, propionitrile, THF and ethylacetate.

The cyanide exchange reaction may also be performed in an ionic liquid of the general formula (R″)₄N⁺, Y⁻, wherein R″ are alkyl-groups or two of the R″ groups together form a ring and Y⁻ is the counterion. In one embodiment of the invention, (R″)₄N⁺Y⁻ represents

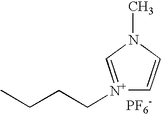

In still another alternative, the cyanide exchange reaction is conducted with apolar solvents such as benzene, xylene or mesitylene and under the influence of microwaves by using i.e. Synthewave 1000™ by Prolabo.

The temperature ranges are dependent upon the reaction type. If no catalyst is present, preferred temperatures are in the range of 100-200° C. When the reaction is conducted under the influence of microwaves, the temperature in the reaction mixture may raise to above 300° C. More preferred temperature ranges are between 120-170° C. The most preferred range is 130-150° C.

If a catalyst is present, the preferred temperature range is between 0 and 100° C. More preferred are temperature ranges of 40-90° C. Most preferred temperature ranges are between 60-90° C.

Other reaction conditions, solvents, etc. are conventional conditions for such reactions and may easily be determined by a person skilled in the art.

Another process for the conversion of a compound of formula (IV), wherein X is Br to the corresponding 5-cyano derivative involves reaction of 5-Br-citalopram of formula (IV) with magnesium to form a Grignard reagent, followed by reaction with a formamide to form an aldehyde. The aldehyde is converted to an oxime or a hydrazone which is converted to a cyano group by dehydration and oxidation, respectively.

Alternatively, 5-Br-citalopram of formula (IV), wherein X is bromo, may be reacted with magnesium to form a Grignard reagent, followed by reaction with a compound containing a CN group bound to a leaving group.

A detailed description of the above two procedures may be found in WO 01/02383.

Compounds of formula (IV), wherein the group X is —CHO, may be converted to escitalopram by methods analogous to those described in WO 99/30548.

Compounds of formula (IV), wherein the group X is NHR¹, wherein R¹ is hydrogen or alkylcarbonyl may be converted by to escitalopram methods analogous to those described in WO 98/19512.

Compounds of formula (IV), wherein the group X is —CONR²R³, wherein R² and R³ are selected from hydrogen optionally substituted alkyl, aralkyl or aryl, may be converted to escitalopram by methods analogous to those described in WO 98/19513 and WO 98/19511.

Compounds of formula (IV), wherein the group X is a group of formula (X), may be converted to escitalopram by methods analogous to those described in WO 00/23431.

Compounds of formula (IV), wherein X is OH, —CH₂OH, —CH₂NH₂, —CH₂NO₂, —CH₂Cl, —CH₂Br, —CH₃ and any of the other groups X above, may be converted to escitalopram by methods analogous to those prepared in WO 01/168632.

Starting materials of formulas (V) and (VI) may be prepared according to the above mentioned patents and patent applications or by analogous methods.

Thus the acid addition salts used according to the invention may be obtained by treatment of intermediates for the synthesis of escitalopram with the acid in a solvent followed by precipitation, isolation and optionally re-crystallisation by known methods and, if desired, micronisation of the crystalline product by wet or dry milling or another convenient process or preparation of particles from a solvent-emulsification process.

In the following, the invention is illustrated by way of examples. However, the examples are merely intended to illustrate the invention and should not be construed as limiting.

### EXAMPLE 1

### Separation of the enantiomers of 4-[4-(dimethylamino)-1-(4′-fluorophenyl)-1-hydroxy-1-butyl]-3-(hydroxymethyl)-benzonitrile

4-[4-(dimethylamino)-1-(4′-fluorophenyl)-1-hydroxy-1-butyl]-3-(hydroxymethyl)-benzonitrile, which may be manufactured according to U.S. Pat. No. 4,650,884, was separated into its enantiomers as follows.

A Novasep Licosep™ 10-50 Simulated Moving Bed Chromatograph was fitted with eight 50 mm i.d. columns each packed to a bed length of 15 cm with Chiralpak™ AD (20 μm) packing material using standard techniques. A SMB system of 8 columns in a 2-2-2-2 configuration was chosen for this separation. Acetonitrile (Baker HPLC grade) was used as mobile phase.

The SMB operating conditions were:
Temperature: 30° C.
Feed Flow (65 mg/mL): 10 mL/min
Eluent Flow (make-up): 102 mL/min
Extract Flow: 69 mL/min
Raffinate Flow: 48 mL/min
Recycle Flow: 210 mL/min
Switch Time: 1.18 min

The products were isolated from the eluent by evaporation resulting in viscous oils. Both enantiomers were isolated with a purity exceeding 99% ee.

The obtained (S)-4-[4-(dimethylamino)-1-(4′-fluorophenyl)-1-hydroxy-1-butyl]-3-(hydroxymethyl)-benzonitrile may be transformed into escitalopram by methods known to those skilled in the art, such as treatment with para-toluensulfonylchloride and a base, e.g. triethylamine, as disclosed in U.S. Pat. No. 4,943,590.

### EXAMPLE 2

### Separation of 1-(4-bromo-2-hydroxymethyl-phenyl)-4-dimethylamino-1-(4-fluorophenyl)-butan-1-ol

A column with the dimensions 280×110 mm packed with ChiralPak® (20 μm particle size) was used as the chiral stationary phase. A mixture of 95% acetonitrile and 5% methanol was used as the mobile phase.

The operation conditions were as follows:
Temperature: 29° C.
Flow rate: 500 mL/min
Detection: UV 280 nm

500 g of a crude citalopram product containing 89% racemate was separated on the column. The first eluting enantiomer with a retention time of 11.0 min was isolated from the eluent with an enantiomeric excess of 99.5% in 99% yield. The second eluting enantiomer with a retention time of 14.1 min was isolated from the eluent with an enantiomeric excess of 99.2% in 98% yield.

### EXAMPLE 3

### Separation of 1-(4′-fluorophenyl)-1-(3-dimethylaminopropyl)-5-bromophtalane into its enantiomers.

A column with the dimensions 280×110 mm packed with Chiralcel®OD (20 μm particle size) was used as the chiral stationary phase. A mixture of 98% vol isohexane and 2% vol isopropanol was used as the mobile phase.

The operation conditions were as follows:
Temperature: Ambient temperature
Flow rate: 500 mL/min
Detection: UV 285 nm

500 g of a crude product containing 89% racemate was separated on the column. The first eluting enantiomer with a retention time of 5.4 min was isolated from the eluent with an enantiomeric excess of 99.5% in 96% yield. [α]_{D}=−0.81° (c=0.99, MeOH); The second eluting enantiomer with a retention time of 6.7 min was isolated from the eluent with an enantiomeric excess of 99.4% in 99% yield. [α]_{D}=+0.95° (c=1.26, MeOH);

### EXAMPLE 4

### Separation of 1-(4′-fluorophenyl)-1-(3-dimethylaminopropyl)-5-bromophtalane into its enantiomers using supercriticalfluid chromatography.

A column with the dimensions 250×10 mm packed with Chiralcel®OD (10 μm particle size) was used as the chiral stationary phase. As mobile phase was used carbon dioxide and modifier in a ratio of 90:10. The modifier was methanol with diethylamine (0.5%) and trifluoroacetic acid (0.5%).

The operation conditions were as follows:
Temperature: Ambient temperature
Flow rate: 18.9 mL/min
Pressure: 20 kPa
Detection: UV 254 nm

75 mg of racemic mixture was separated on the column.

Both enantiomers were isolated from the eluent. The enantiomers were isolated with an enatiomeric excess of 86.1% (RT 3.25 min) and 87.1% (RT 3.67 min), respectively.

### EXAMPLE 5

### Cyanation of (+)-1-(4′-fluorophenyl)-1-(3-dimethylaminopropyl)-5-bromophtalane.

5.0 g of the (+)-enantiomer was treated with 3.1 g of Zn(CN)₂ and 0.76 g of Pd(PPh₃)₄ under the conditions described in the WO 00/13648. The enantiomeric purity of the product was analysed by chiral electrophoresis. Based on the results from chiral electrophoresis and supercritical fluid chromatography, the product was shown to be identical with escitalopram. Yield: 80%; ee 99.6%

1. Method for preparation of escitalopram having the formula 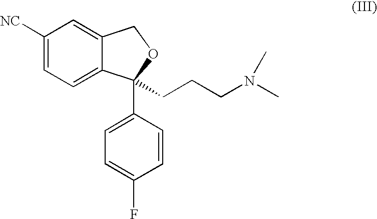
or pharmaceutically acceptable addition salts thereof comprising separation of the enantiomers of a compound selected from the group comprising citalopram having the formula 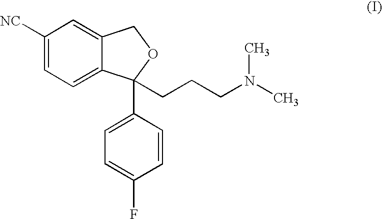
and intermediate compounds in the preparation of citalopram, characterised in that said separation of enantiomers is performed by liquid chromatographic separation of enantiomers using a chiral stationary phase for the chromatography.

2. A method according to claim 1 comprising preparation of a compound of formula 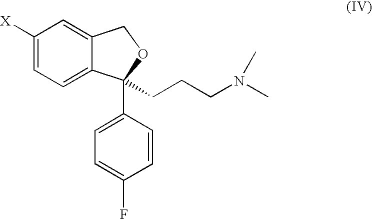
wherein X is a cyano group or halogen or any other group that may be converted to a cyano group, by optical resolution by chromatography of a racemic compound of formula 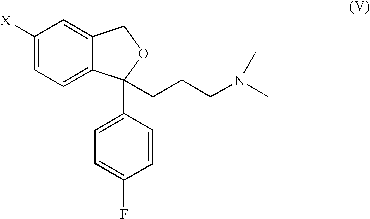
wherein X is as defined above, and if X is not a cyano group then followed by conversion of the group X in the compound of formula (IV) to a cyano group followed by isolation of escitalopram or a pharmaceutically acceptable salt thereof.

3. Method according to claim 2, wherein the group X is cyano.

4. The method according to claim 2, wherein the group X is bromo.

5. A method according to claim 1 comprising optical resolution by chromatography of a compound of formula 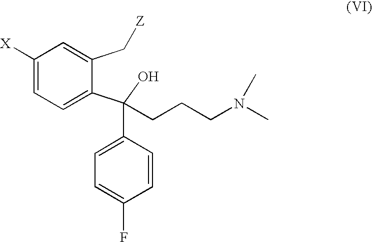
wherein X is a cyano group or halogen or any other group that may be converted to a cyano group and Z is hydroxy or a leaving group, to form the compound of formula 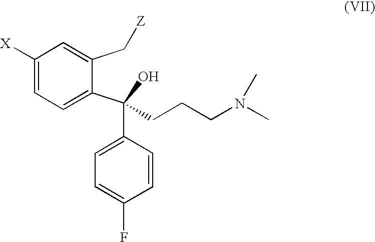
and if Z is OH conversion of the group Z to a leaving group and then ring closure of the resulting compound of formula (VII) wherein Z is a leaving group to form a compound of formula 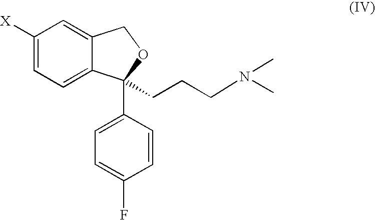
wherein X is as defined above, and if X is not a cyano group then conversion of the group X in the compound of formula (IV) to a cyano group, followed by isolation of escitalopram or a pharmaceutically acceptable salt thereof.

6. Method according to claim 5, wherein the group X is cyano.

7. Method according to claim 5, wherein the group X is bromo.

8. The method according to claim 1, characterised in that the chiral stationary phase comprises a carbohydrate derivative.

9. Method according to claim 8, characterised in that the carbohydrate derivative is a polysaccharide derivative.

10. The method according to claim 8, characterised in that the carbohydrate derivative comprises phenyl carbamate substituents which optionally may be substituted with one or more C₁₋₄-alkyl groups, preferably methyl groups.

11. The method according to claim 9, characterised in that the polysaccharide derivative is an amylose derivative.

12. Method according to claim 11, characterised in that the chiral stationary phase comprising an amylose derivative comprising optionally alkyl substituted phenyl carbamate substituents is a silica gel supported amylose derivative wherein the majority of the hydroxyl groups are substituted with 3,5-dimethylphenyl carabamate groups.

13. The method according to claim 9, characterised in that the polysaccharide derivative is a cellulose derivative.

14. Method according to claim 13, characterised in that the chiral stationary phase comprising a cellulose derivative comprising optionally alkyl substituted phenyl carbamate substituents is a silica gel supported cellulose derivative wherein the majority of the hydroxyl groups are substituted with 3,5-dimethylphenyl carbamate groups.

15. The method according to claim 8, characterised in that the carbohydrate derivative is adsorbed on silica gel.

16. The method according to claim 1, characterised in that the chromatographic separation comprises a continuous chromatographic process, suitably Simulated Moving Bed technology.

17. The method according to claim 1, wherein a compound of formula(III), wherein X is halogen, in particular bromo, is converted to escitalopram by reaction of the compound of formula (IV) with CuCN followed by purification and isolation of escitalopram or a pharmaceutically acceptable salt thereof.

18. The method according to claim 1, wherein the compound of formula (IV), wherein X is halogen, in particular bromo, or CF₃—(CF₂)ₙ—SO₂—O—, wherein n is 0-8, is converted to escitalopram by reaction of the compound of formula (III) with a cyanide source in presence of a palladium catalyst followed by purification and isolation of escitalopram or a pharmaceutically acceptable salt thereof.

19. The method according to claim 1, wherein a compound of formula (IV) wherein X is halogen, in particular bromo, is converted to escitalopram by reaction of a compound of formula (III) with a cyanide source in presence of a nickel catalyst followed by purification and isolation of escitalopram or a pharmaceutically acceptable salt thereof.

20. An intermediate having the formula 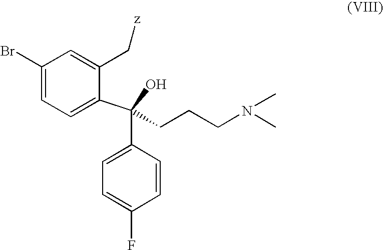
wherein Z is hydroxy or a leaving group; or a salt thereof.

21. (canceled)

22. The method according to claim 9, wherein the carbohydrate derivative comprises phenyl carbamate substituents which optionally may be substituted with one or more C₁₋₄-alkyl groups, preferably methyl groups.

23. The method according to claim 10, wherein the polysaccharide derivative is an amylose derivative.

24. The method according to claim 22, wherein the polysaccharide derivative is an amylose derivative.

25. The method according to claim 23, wherein the chiral stationary phase comprising an amylose derivative comprising optionally alkyl substituted phenyl carbamate substituents is a silica gel supported amylose derivative wherein the majority of the hydroxyl groups are substituted with 3,5-dimethylphenyl carabamate groups.

26. The method according to claim 24, wherein the chiral stationary phase comprising an amylose derivative comprising optionally alkyl substituted phenyl carbamate substituents is a silica gel supported amylose derivative wherein the majority of the hydroxyl groups are substituted with 3,5-dimethylphenyl carabamate groups.

27. The method according to claim 10, wherein the polysaccharide derivative is a cellulose derivative.

28. The method according to claim 22, wherein the polysaccharide derivative is a cellulose derivative.
